(19) United States
(12) Patent Application Publication
(10) Pub. No.: US 2025/0057747 A1
(43) Pub. Date: Feb. 20, 2025

(54) ORGANIC COMPOUNDS AS FRAGRANCE

(71) Applicant: GIVAUDAN SA, VERNIER (CH)

(72) Inventors: Nicolas COCITO ARMANINO, Kemptthal (CH); Chao WANG, Shanghai (CN)

(21) Appl. No.: 18/712,550

(22) PCT Filed: Nov. 21, 2022
(86) PCT No.: PCT/EP2022/082552

§ 371 Date: May 22, 2024

(30) Foreign Application Priority Data
Nov. 22, 2021 (WO) PCT/CN2021/132118

(51) Int. Cl. A61K 8/49; A61Q 13/00; C07D 313/00; C11D 3/20; C11D 3/50

(52) U.S. Cl. CPC A61K 8/4973; A61Q 13/00; C07D 313/00; C11D 3/2096; C11D 3/50

(57) ABSTRACT

Disclosed are oxa macrocyclic ketones of formula (I) possessing musky, powdery, nitro musk odor characteristics.

The present invention is concerned with fragrance ingredients and with fragrance preparations, for imparting desired odor notes to consumer products, in particular it is concerned with a novel class of oxa macrocyclic ketones of formula (I) possessing musky, powdery, nitro musk odor characteristics.

In the fragrance and flavour industry, perfumers and flavorists are continually looking for new compounds possessing unique odor characteristics.

Compounds having musk odor characteristics are well known in the art. The best known commercially established cyclic compounds possessing musk odor characteristics are selected from the chemical class of macrocyclic ketones, such as Muscenone (3-methylcyclopentadec-5-en-1-one), lactones, such as Exaltolid™ (16-oxacyclohexadecan-1-one) or Nirvanolide® (13-Methyloxacyclopentadec-10-en-2-one) and oxalactones, such as Musk R1® (1,7-dioxacycloheptadecan-8-one).

However, there are hardly any oxacyclic ketones known in the art. This class of compounds is described in literature mainly as intermediates, e.g. for the preparation of macrocyclic lactones (K. C. Nicolaou et al., J. Am. Chem. Soc. 1998, 120, 5132-5133). As a further example one may cite the German patent application DE10163579 A1, describing a process for the oxidation, inter alia, of oxacycloalkane derivatives to oxacycloalkandione derivatives (e.g. 1-oxacyclohexadecan-5-on to 1-oxacyclohexadecan-2, 13-dion), using a liquid multi-phase catalyst system comprising at least one ruthenium-containing compound and an oxygen atom.

There is only one document from 1936 with the title “many-membered cyclic oxides and oxido ketones” (Stoll et al., Helv. Chim. Acta, 1936 (19), 735-743) disclosing some odor properties. The only oxido ketone disclosed is 1,15-oxido-pentadecanon-5(oxacyclohexadecan-6-one) an isomer of Exaltolid™. According to the authors the odor properties of oxacyclohexadecan-6-one totally changed compared to Exaltolid™. In particular oxacyclohexadecan-6-one exhibits a strong sweet note with some fatty nuances and partially reminiscent of vanillin, even though the musk odor was maintained.

The primary objective of the present invention was to find new substances that possess interesting odor characteristics and are suitable as fragrances for use in perfumery. In particular, musky, nitro musk notes lacking sweet odor characteristics.

Compounds possessing the typical nitro musk character, such as Musk Keton™ (1-(4-tert-butyl-2,6-dimethyl-3,5-dinitrophenyl)ethanone), are restricted in use. Thus there is a particular interest in novel compounds possessing the sought after odor reminiscent to nitro musk.

Thus there is provided in accordance with a first aspect of the present invention the use as fragrance of a compound of formula (I)

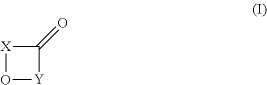

wherein
X is a divalent residue —(CH2)ₙ— optionally substituted with one additional methyl group,
Y is divalent residue —(CH2)ₘ—,
n is an integer selected from 3 to 12, and
m is an integer selected from 3 to 12,
with the proviso that the n+m is at least 13 and not greater than 15, e.g. n+m is 14; and
with the proviso that the compound of formula (I) is not oxacyclohexadecan-6-one.

In another particular embodiment there is provided the use as fragrance of a compound of formula (I) wherein
X is a divalent residue —(CH2)ₙ— substituted with one additional methyl group,
Y is divalent residue —(CH2)ₘ—,
n is an integer selected from 3 and 4, and
m is an integer selected from 9 to 11,
with the proviso that the n+m is at least 13 and not greater than 15, e.g. n+m is 14.

In another particular embodiment there is provided the use as fragrance of a compound of formula (I) wherein
X is a divalent residue —(CH2)ₙ—,
Y is divalent residue —(CH2)ₘ—,
n is an integer selected from 3 to 7, and
m is an integer selected from 7 to 12,
with the proviso that the n+m is at least 13 and not greater than 15, e.g. n+m is 14; and
with the proviso that the compound of formula (I) is not oxacyclohexadecan-6-one.

In another particular embodiment there is provided the use as fragrance of a compound of formula (I) wherein
X is a divalent residue —(CH2)ₙ—,
Y is divalent residue —(CH2)ₘ—,
n is an integer selected from 6, 7 and 8, and
m is an integer selected from 6, 7 and 8,
with the proviso that n+m is at least 13 and not greater than 15, e.g. n+m is 14.

In another particular embodiment there is provided the use as fragrance of a compound of formula (I) wherein
X is a divalent residue —(CH2)ₙ—,
Y is divalent residue —(CH2)ₘ—,
n is an integer selected from 6 and 7, and
m is an integer selected from 6 to 9,
with the proviso that n+m is at least 13 and not greater than 15, e.g. n+m is 14.

In another embodiment there is provided the use as fragrance of a compound of formula (I) selected from the group consisting of 4-methyloxacyclopentadecan-6-one; 3-methyloxacyclopentadecan-5-one; oxacyclopentadecan-5-one; oxacyclopentadecan-6-one; oxacyclopentadecan-7-one; oxacyclopentadecan-8-one; 3-methyloxacyclohexadecan-5-one; 4-methyloxacyclohexadecan-6-one; oxacyclohexadecan-5-one; oxacyclohexadecan-7-one; oxacyclohexadecan-8-one; oxacycloheptadecan-5-one; oxacycloheptadecan-6-one; oxacycloheptadecan-7-one; oxacycloheptadecan-8-one; and oxacycloheptadecan-9-one.

The compounds of formula (I) wherein X is a divalent residue substituted with one methyl group comprises one chiral centre and as such may exist as a mixture of stereoisomers, or it may be resolved as isomerically pure form. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methodology known in the art, e.g. preparative HPLC and GC or by stereoselective syntheses.

The compounds of formula (I) may be used alone, as isomeric mixture thereof, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, and other auxiliary agents commonly used in the art.

As used herein, “carrier material” means a material which is practically neutral from a odorant point of view, i.e. a material that does not significantly alter the organoleptic properties of odorants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a fragrance composition. A detailed description of the nature and type of adjuvants commonly used in fragrance compositions containing same cannot be exhaustive, but it has to be mentioned that said ingredients are well known to a person skilled in the art.

As used herein, ‘fragrance composition’ means any composition comprising a compound of formula (I), or a mixture thereof and a base material, e.g. a diluent conventionally used in conjunction with odorants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following list comprises examples of known odorant molecules, which may be combined with a compound of formula (I), or a mixture thereof:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super@(1-(2,3,8,8-tetramethyl-1,2,3,4,5,6,7,8-octahydronaphthalen-2-yl)ethanone); Isoraldeine@((E)-3-methyl-4-(2,6,6-trimethylcyclohex-2-en-1-yl)but-3-en-2-one); Hedione™ (methyl 3-oxo-2-pentylcyclopentaneacetate); 3-(4-isobutyl-2-methylphenyl)propanal; maltol; methyl cedryl ketone (1-((3R,3AR,7R,8aS)-3,6,8,8-tetramethyl-2,3,4,7,8,8a-hexahydro-1H-3a,7-methanoazulen-5-yl)ethan-1-one); methylionone; verbenone; and/or vanillin;
ether and acetals, e.g. Ambrox@(3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); and/or Spirambrene@(2′,2′,3,7,7-pentamethylspiro[bicyclo[4.1.0]heptane-2,5′-[1,3]dioxane]);
esters and lactones, e.g. benzyl acetate; cedryl acetate ((1S,6R,8aR)-1,4,4,6-tetramethyloctahydro-1H-5,8a-methanoazulen-6-yl acetate); delta-decalactone (6-pentyltetrahydro-2H-pyran-2-one); Helvetolide@(2-(1-(3,3-dimethylcyclohexyl)ethoxy)-2-methylpropyl propionate); delta-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate);
macrocycles, e.g. Ambrettolide ((Z)-oxacycloheptadec-10-en-2-one); (E)-oxacycloheptadec-10-en-2-one; ethylene brassylate (1,4-dioxacycloheptadecane-5,17-dione); and/or Exaltolide@(16-oxacyclohexadecan-1-one); and
heterocycles, e.g. isobutylquinoline (2-isobutylquinoline).

Thus there is provided in a further aspect of the invention a fragrance composition comprising a compound of formula (I) and at least one other odorant molecule.

The compounds of formula (I) may be used in a broad range of fragranced articles, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion is typically from 0.00001 to 3 weight percent of the article. In one embodiment, the compound may be employed in a fabric softener in an amount from 0.0001 to 0.3 weight percent (e.g. 0.001 to 0.1 including 0.05 weight %). In another embodiment, the compound may be used in fine perfumery in amounts from 0.01 to 30 weight percent (e.g. up to about 10 or up to 20 weight percent), more preferably between 0.01 and 5 weight percent (e.g. 0.01 to 0.1 weight percent). However, these values are given only by way of example, since the experienced perfumer may also achieve effects or may create novel accords with lower or higher concentrations.

The compounds of formula (I) may be employed in a consumer product base simply by directly mixing the compound, or a fragrance composition comprising a compound of formula (I), or a mixture thereof, with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, the invention additionally provides a method of manufacturing a fragranced article, comprising the incorporation a compound of formula (I), or a mixture thereof as a fragrance ingredient, either by directly admixing to the consumer product base or by admixing a fragrance composition comprising a compound of formula (I), or a mixture thereof, which may then be mixed with a consumer product base, using conventional techniques and methods.

Through the addition of an olfactory acceptable amount of a compound of formula (I), or a mixture thereof the odor notes of a consumer product base will be improved, enhanced, or modified.

Thus, the invention furthermore provides a method for improving, enhancing or modifying a consumer product base by means of the addition thereto of an olfactorily acceptable amount of a compound of formula (I), or a mixture thereof.

There is provided in a further aspect of the present invention a fragranced article comprising:
a) a compound of formula (I)

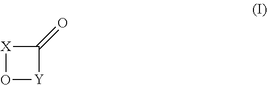

wherein
   X is a divalent residue —(CH2)ₙ— optionally substituted with one additional methyl group,
   Y is divalent residue —(CH2)ₘ—,
   n is an integer selected from 3 to 12, and
   m is an integer selected from 3 to 12,
   with the proviso that the n+m is at least 13 and not greater than 15, e.g. n+m is 14; and
b) a consumer product base.

As used herein, ‘consumer product base’ means a composition for use as a consumer product to fulfill specific actions, such as cleaning, softening, and caring or the like. Examples of such products include fine perfumery, e.g. perfume and eau de toilette; fabric care, household products and personal care products such as cosmetics, laundry care detergents, rinse conditioner, personal cleansing composition, detergent for dishwasher, surface cleaner; laundry products, e.g. softener, bleach, detergent; body-care products, e.g. shampoo, shower gel; air care products (includes products that contain preferably volatile and usually pleasant-smelling compounds which advantageously can even in very small amounts mask unpleasant odors). Air fresheners for living areas contain, in particular, natural and synthetic essential oils such as pine needle oils, citrus oil, eucalyptus oil, lavender oil, and the like, in amounts for example of up to 50% by weight. As aerosols they tend to contain smaller amounts of such essential oils, by way of example less than 5% or less than 2% by weight, but additionally include compounds such as acetaldehyde (in particular, <0.5% by weight), isopropyl alcohol (in particular, <5% by weight), mineral oil (in particular, <5% by weight), and propellants.

Cosmetic products include:
(a) cosmetic skincare products, especially bath products, skin washing and cleansing products, skincare products, eye makeup, lip care products, nail care products, intimate care products, foot care products;
(b) cosmetic products with specific effects, especially sunscreens, tanning products, de-pigmenting products, deodorants, antiperspirants, hair removers, and shaving products;
(c) cosmetic dental-care products, especially dental and oral care products, tooth care products, cleaners for dental prostheses, adhesives for dental prostheses; and
(d) cosmetic hair care products, especially hair shampoos, hair care products, hair setting products, hair-shaping products, and hair coloring products.

This list of products is given by way of illustration, and is not to be regarded as being in any way limiting.

In one particular embodiment the consumer product base is selected form fine perfumery, and personal care products, including deodorants, hair care products, soaps, and the like.

In a further particular embodiment the consumer product base is selected from fabric care products, including fabric softener, and home care products, including air fresheners, dish washers and the like.

Most of the compounds of formula (I) are novel in its own. Thus there is provided in accordance with a further aspect of the present invention compounds of formula (I)

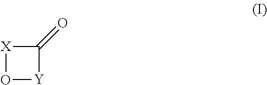

wherein
X is a divalent residue —(CH2)ₙ— optionally substituted with one additional methyl group,
Y is divalent residue —(CH2)ₘ—,
n is an integer selected from 3 to 7, and
m is an integer selected from 8 to 11,
with the proviso that the n+m is at least 13 and not greater than 15 (e.g. n+m is 4); and
with the proviso that oxacyclohexadecan-6-one and oxacyclohexadecan-5-one are excluded.

The compounds of formula (I) may, for example, be synthesized from the respective oxa-diacids or oxa-diesters by Dieckmann reaction and in-situ decarboxylation. The oxa-diesters or oxa-diacids can be obtained by oxidation of the corresponding primary oxa-diols (e.g. by reaction with chromium trioxide in sulfuric acid). Synthesis of the ether bond may be effected by reacting a mono-protected (e.g. benzyl protected) diol with a mono-protected (e.g. benzyl protected), mono-activated (e.g. toluenesulfonyl) diol. Following formation of the ether bond, a deprotection furnishes the desired oxa-diol.

The compounds of formula (I) wherein X is a divalent residue substituted with one methyl group may, for example, also be prepared by converting the suitable, unsaturated ethers to the respective diester by hydro-carbonylation, followed by Dickemann reaction and in-situ decarboxylation.

The invention is now further described with reference to the following non-limiting examples. These examples are for the purpose of illustration only and it is understood that variations and modifications can be made by one skilled in the art.

### Example 1: 4-methyloxacyclohexadecan-6-one

Example 1a: 10-((3-methylbut-3-en-1-yl)oxy)dec-1-ene: A suspension of sodium hydride (0.65 g, 60 wt % in mineral oil, 16.2 mmol) in tetrahydrofuran (THF) (30 mL) was treated slowly at room temperature (r.t.) with a solution of 3-methylbut-3-en-1-ol (1.20 g, 1.41 mL, 97 wt %, 13.5 mmol) in THF (10 mL). After addition the mixture was heated to 60° C. and stirred for 1 h and then treated with 10-bromodec-1-ene (3.55 g, 16.2 mmol) and the resulting mixture stirred at 60° C. overnight. After cooling to r.t., the mixture was poured onto iced water (50 mL), extracted with MTBE (methyl tert-butyl ether) (2×80 mL), washed with water (80 mL) and brine (50 mL), dried over Na₂SO₄, filtered and concentrated. The resulting material was purified by flash column chromatography on silica gel, eluting with a gradient of MTBE in heptane to give 10-((3-methylbut-3-en-1-yl)oxy)dec-1-ene (1.6 g, 7.13 mmol, 53% yield) as a yellow liquid.

1H NMR (400 MHz, CDCl3, 298 K) δ (ppm)=5.83 (tdd, J=6.6, 10.3, 17.1 Hz, 1H), 5.01 (qd, J=1.8, 17.1 Hz, 1H), 4.95 (tdd, J=1.2, 2.3, 10.2 Hz, 1H), 4.82-4.72 (m, 2H), 3.54 (t, J=7.1 Hz, 2H), 3.44 (t, J=6.7 Hz, 2H), 2.32 (t, J=7.0 Hz, 2H), 2.10-2.02 (m, 2H), 1.77 (s, 3H), 1.59 (quin, J=7.0 Hz, 2H), 1.45-1.26 (m, 10H).

13C NMR (101 MHz, CDCl3, 298 K) δ (ppm)=143.0, 139.2, 114.1, 111.3, 71.0, 69.3, 37.8, 33.8, 29.7, 29.4, 29.4, 29.1, 28.9, 26.2, 22.8.

GC/MS (El, 70 eV): 209 (2, [M-CH₃]⁺), 169 (1), 155 (1), 109 (9), 99 (36), 83 (74), 70 (100), 69 (63), 55 (95).

Example 1b: methyl 11-((5-methoxy-3-methyl-5-oxopentyl)oxy)undecanoate: In an autoclave, a solution of 10-((3-methylbut-3-en-1-yl)oxy)dec-1-ene (1.60 g, 7.13 mmol) in Methanol (15 mL) was treated with Bis(acetonitrile)dichloropalladium(II) (93.4 mg, 99 wt %, 357 μmol), (9,9-dimethyl-9H-xanthene-4,5-diyl)bis(diphenylphosphane) (213 mg, 97 wt %, 357 μmol) and methanesulfonic acid (69.2 mg, 99 wt %, 713 μmol) and the resulting mixture flushed with carbon monoxide (CO) then pressurized with CO to 15 bar and heated to 80° C. for 20 h. The mixture was then cooled to r.t., the gas pressure was released carefully and the mixture poured into iced water (100 mL), extracted with EtOAc (ethyl acetate) (2×50 mL), washed with water (50 mL), brine (50 mL), dried over Na₂SO₄, filtered and concentrated. The resulting material was purified by flash column chromatography on silica gel, eluting with a gradient of MTBE in heptane to give methyl 11-((5-methoxy-3-methyl-5-oxopentyl)oxy)undecanoate (1.40 g, 3.43 mmol, 48% yield) along with other regioisomers, as a pale yellow oil which was used in the following step without further purification.

GC/MS (El, 70 eV): 344 (1, [M]⁺*), 312 (1), 280 (2), 271 (2), 215 (12), 199 (4), 183 (20), 145 (61), 129 (75), 113 (100) 101 (22), 97 (25), 87 (49), 69 (66), 59 (21), 55 (41), 41 (25).

Example 1c: mixture of methyl 4-methyl-6-oxooxacyclohexadecane-7-carboxylate and methyl 4-methyl-6-oxooxacyclohexadecane-5-carboxylate: A solution of methyl 11-((5-methoxy-3-methyl-5-oxopentyl)oxy)undecanoate (1.40 g, 4.06 mmol) in THF (180 ml) was added dropwise over 6.5 h to a refluxing mixture of NaHMDS (sodium hexamethyldisilazide) (8.94 g, 24.4 mL, 2.0 molar in THF, 48.8 mmol) and THF (120 ml). The reaction was then further refluxed for additional 30 min then cooled to r.t. and slowly treated with acetic acid (AcOH) (20 mL). The mixture was then washed with water (3×150 mL) and the washings extracted with MTBE (200 mL). the combined organic layers were washed with brine (150 mL), dried over Na₂SO₄, filtered and concentrated. The resulting material was purified by flash column chromatography on silica gel, eluting with a gradient of MTBE in heptane to give a mixture of methyl 4-methyl-6-oxooxacyclohexadecane-7-carboxylate and methyl 4-methyl-6-oxooxacyclohexadecane-5-carboxylate (0.190 g, 89% purity, 13% yield) which was taken to the next step without further purification.

GC/MS (El, 70 eV): 312 (1, [M]⁺*), 281 (3), 170 (5), 115 (100).

Example 1d: 4-methyloxacyclohexadecan-6-one: A solution of mixture of methyl 4-methyl-6-oxooxacyclohexadecane-7-carboxylate and methyl 4-methyl-6-oxooxacyclohexadecane-5-carboxylate (0.17 g, 0.54 mmol) in MeOH (5 ml) was treated with aqueous NaOH (0.82 mL, 2 molar, 1.6 mmol) and the resulting mixture heated to reflux (80° C.) for 1 h. The resulting mixture was then cooled to r.t., acidified to pH 1 by dropwise addition of 10% H₂SO₄ and then heated to reflux for 30 min. The mixture was then cooled to r.t., extracted with MTBE (2×50 mL), washed with water (50 mL), brine (50 mL), dried over Na₂SO₄, filtered and concentrated. The resulting material was purified by flash column chromatography on silica gel, eluting with a gradient of MTBE in heptane to give 4-methyloxacyclohexadecan-6-one (0.100 g, 98% purity, 0.54 mmol, 71% yield) as a colorless liquid.

1H NMR (400 MHz, CDCl3, 298 K) δ (ppm)=3.53-3.35 (m, 4H), 2.57 (dd, J=5.9, 14.1 Hz, 1H), 2.49-2.35 (m, 2H), 2.34-2.21 (m, 1H), 2.20-2.11 (m, 1H), 1.76-1.21 (m, 18H), 0.93 (d, J=6.6 Hz, 3H).

13C NMR (101 MHz, CDCl3, 298 K) δ (ppm)=212.2, 70.3, 68.0, 50.7, 40.7, 36.6, 29.1, 27.1, 27.0, 26.9, 26.8, 25.9, 25.8, 25.0, 22.4, 20.0.

GC/MS (El, 70 eV): 254 (6, [M]+*), 239 (1), 167 (4), 149 (6), 125 (37), 115 (63), 97 (100), 83 (26), 69 (53), 55 (63), 41 (54).

Odour description (10% solution in DPG on paper blotter, 4 h): musky, powdery, ambery, nitro musk.

### Example 2: oxacyclopentadecan-6-one

Example 2a: methyl 10-((5-methoxy-5-oxopentyl)oxy)decanoate: A mixture of 1,7-dioxacycloheptadecan-8-one (58.33 g, 228 mmol) and NaOH (10.94 g, 273 mmol) in water (100 mL) was refluxed for 4 hours. After the total conversion of the starting lactone (monitoring with GC and TLC), the mixture was cooled to room temperature, then diluted HCl was added dropwise to obtain pH=6-7. The mixture was then cooled to 0° C., then KMnO₄ (43.20 g, 273 mmol) was added in portions to the reaction mixture at 0° C. and the resulting mixture stirred overnight at room temperature. The mixture was then filtered and the filtrate treated dropwise with 2M HCl to obtain pH=4-5 with concomitant formation of a white precipitate. The precipitate was filtered to give crude diacid (70.01 g) as a white solid. The solid was mixed with methanol (100 mL) and treated with 2 drops of conc. sulfuric acid. The reaction mixture was stirred at reflux for 2 hours then cooled to room temperature. The solvent was evaporated and the resulting oil purified by column chromatography on silica gel, eluting with a gradient of MTBE in heptane to give methyl 10-((5-methoxy-5-oxopentyl)oxy)decanoate (19.37 g, 61 mmol, 27% yield) as a colorless oil.

1H NMR (400 MHz, CDCl3) δ (ppm)=3.68, 3.66 (s, 6H), 3.52-3.20 (m, 4H), 2.37-2.25 (m, 4H), 1.81-1.42 (m, 8H), 1.38-1.22 (m, 10H).

13C NMR (101 MHz, CDCl3) δ (ppm)=174.23 (s), 173.99 (s), 70.96 (t), 70.23 (t), 51.40 (q), 51.36 (q), 34.05 (t), 33.78 (t), 29.70 (t), 29.37 (t), 29.33 (t), 29.14 (t), 29.08 (t), 26.12 (t), 24.90 (t), 21.75 (t).

GC/MS (El): m/z (%): 284 (1) [M+], 252 (4), 201 (17), 169 (42), 131 (77), 115 (94), 101 (86), 99 (97), 83 (65), 73 (50), 55 (100).

Example 2b: mixture of methyl 6-oxooxacyclopentadecane-7-carboxylate and methyl 6-oxooxacyclopentadecane-5-carboxylate: A solution of methyl 10-((5-methoxy-5-oxopentyl)oxy)decanoate (6.00 g, 17 mmol) in THF (340 mL) was slowly added at a speed of 50 mL/h to a refluxing mixture of LiHMDS (Lithium hexamethyldisilazide) (180 mL, 1M in THF, 180 mmol) and THF (220 mL) under Argon. After completion of the feed, the mixture was further stirred for 15 min at reflux. The mixture was then cooled to room temperature and treated with diluted HCl to obtain a pH=4-5, extracted with MTBE (3×200 mL), washed with water (200 mL), brine (200 mL), dried over MgSO₄ and concentrated. The obtained crude oil was purified by column chromatography on silica gel, eluting with a gradient of MTBE in heptane to give a mixture of methyl 6-oxooxacyclopentadecane-7-carboxylate and methyl 6-oxooxacyclopentadecane-5-carboxylate (2.30 g, 8.1 mmol, 43% yield, Ratio of two isomers=1.3:1) as a light yellow oil.

1H NMR (400 MHz, CDCl3) δ (ppm)=3.71 (s, 3H), 3.66-3.57 (m, 1H), 3.54-3.30 (m, 4H), 2.82-2.43 (m, 2H), 2.18-1.86 (m, 2H), 1.85-1.68 (m, 3H), 1.64-1.51 (m, 4H), 1.42-1.28 (m, 9H).

13C NMR (101 MHz, CDCl3) δ (ppm)=207.05 (s), 206.58 (s), 170.34 (s), 170.25 (s), 70.57 (t), 70.33 (t), 70.21 (t), 69.81 (t), 58.40 (d), 57.23 (d), 52.26 (q), 52.22 (q), 42.87 (t), 40.51 (t), 28.92 (t), 28.80 (t), 28.37 (t), 28.09 (t), 27.53 (t), 27.19 (t), 27.00 (t), 26.98 (t), 26.74 (t), 26.41 (t), 26.17 (t), 25.54 (t), 25.40 (t), 25.30 (t), 25.12 (t), 23.23 (t), 21.64 (t).

Major GC/MS (El): m/z (%): 284 (1) [M+], 253 (5), 207 (6), 156 (10), 115 (33), 100 (73), 83 (33), 69 (38), 55 (100)., Minor GC/MS (El): m/z (%): 284 (1) [M+], 253 (3), 207 (1), 156 (10), 110 (9), 101 (100), 83 (36), 69 (16), 55 (65).

Example 2c: oxacyclopentadecan-6-one: A mixture of methyl 6-oxooxacyclopentadecane-7-carboxylate and methyl 6-oxooxacyclopentadecane-5-carboxylate (2.50 g, 8 mmol) was dissolved in MeOH (100 mL) and treated with a solution of NaOH (0.96 g, 24 mmol) in water (100 mL) at room temperature, and the resulting mixture was refluxed for 1 h. After cooling down, the mixture was acidified to pH=3-4 with 10% H₂SO₄, followed by refluxing for 30 min.

The mixture was then cooled to room temperature and concentrated under reduced pressure to remove MeOH. The residue was extracted with MTBE (3×100 mL), washed with water (100 mL), brine (100 mL), dried over MgSO₄ and concentrated. The resulting material was purified by column chromatography on silica gel, eluting with a gradient of MTBE in heptane, followed by Kugelrohr distillation (190° C., 0.082 MPa) to give oxacyclopentadecan-6-one (1.76 g, 7.4 mmol, 92% yield) as a colorless oil. 1H NMR (400 MHz, CDCl3) δ (ppm)=3.49-3.36 (m, 4H), 2.58-2.48 (m, 2H), 2.47-2.37 (m, 2H), 1.79-1.63 (m, 4H), 1.60-1.48 (m, 3H), 1.47-1.20 (m, 11H).

13C NMR (101 MHz, CDCl3) δ (ppm)=213.12 (s), 70.49 (t), 70.18 (t), 43.12 (t), 41.45 (t), 28.97 (t), 28.78 (t), 27.31 (t), 27.05 (t), 26.58 (t), 26.29 (t), 25.12 (t), 24.08 (t), 21.96 (t).

GC/MS (El): m/z (%): 226 (8) [M+], 135 (7), 111 (26), 101 (55), 98 (90), 83 (75), 81 (20), 69 (30), 55 (100).

Odour description (10% solution in DPG on paper blotter, 4 h): musky, powdery, nitro musk.

### Example 3: oxacyclohexadecan-9-one

Example 3a: 8-(benzyloxy)octyl 4-methylbenzenesulfonate: A flask was charged with 8-(benzyloxy)octan-1-ol (10.0 g, 42.3 mmol), Dichloromethane (100 mL), Et₃N (trimethylamine) (5.19 g, 7.15 mL, 50.8 mmol) and the reaction mixture was cooled to 0° C. (ice bath). Finally, p-toluenesulfonyl chloride (9.78 g, 50.8 mmol) was added in small portions over 30 min, the reaction mixture was stirred at 0° C. for 30 min and then stirred for 17 h (overnight) at rt. The reaction mixture was poured into ice-cold HCl 1M (150 ml) and stirred vigorously for 10 min.

The mixture was extracted with MTBE (2×150 ml), washed with sat. NaHCO₃ solution (1×150 ml) and brine (1×100 ml), dried over Na₂SO₄, filtered and concentrated under reduced pressure to give 8-(benzyloxy)octyl 4-methylbenzenesulfonate (17.7 g, 94% purity, 42.5 mmol, quant. yield) as a yellow oil which was used without further purification.

GC/MS (El, 70 eV): 390 (1, [M]⁺*), 372 (1), 218 (1), 173 (33), 155 (6), 107 (37), 91 (100).

Example 3b: (((oxybis(octane-8,1-diyl))bis(oxy))bis(methylene))dibenzene: A suspension of sodium hydride (2.5 g, 60 wt % in mineral oil, 62.2 mmol) in THF (100 ml) was treated at r.t. with a solution of 8-(benzyloxy)octan-1-ol (14.0 g, 59.2 mmol) in THF (50 ml) over 30 min and the resulting reaction mixture heated to 60° C. for 1 h. Finally, a solution of 8-(benzyloxy)octyl 4-methylbenzenesulfonate (24.3 g, 62.2 mmol) in THF (50 ml) was added over 45 min. The reaction mixture was stirred at r.t. for 17 h then at 60° C. for 23 h. The reaction mixture was poured into ice water (200 ml) and stirred vigorously for 10 min. The mixture was then extracted with MTBE (2×200 ml), the org. layers were washed with water (1×200 ml) and brine (1×150 ml). The combined org. layers were dried over Na₂SO₄, filtered and concentrated under reduced pressure to give a product that was purified by column chromatography on silica gel, eluting with a gradient of MTBE in heptane to give (((oxybis(octane-8,1-diyl))bis(oxy))bis(methylene))dibenzene (19.0 g, 41.8 mmol, 71% yield) as a colorless oil.

1H NMR (400 MHz, CDCl3, 298 K) δ (ppm)=7.37-7.30 (m, 8H), 7.30-7.23 (m, 2H), 4.49 (s, 4H), 3.45 (t, J=6.6 Hz, 4H), 3.38 (t, J=6.7 Hz, 4H), 1.65-1.50 (m, 8H), 1.40-1.26 (m, 16H).

13C NMR (101 MHz, CDCl3, 298 K) δ (ppm)=138.7, 128.3, 127.6, 127.5, 72.9, 71.0, 70.5, 29.8, 29.5, 26.2.

GC/MS (El, 70 eV): 363 (5, [M-CH₂Ph]⁺*), 235 (1), 111 (13), 167 (9), 91 (100).

Example 3c: 8,8′-oxybis(octan-1-ol): A solution of (((oxybis(octane-8,1-diyl))bis(oxy))bis(methylene))dibenzene (19.0 g, 41.8 mmol) in Methanol (250 mL) was treated with palladium on carbon (2.22 g, 10 wt % palladium, 2.09 mmol). The flask was placed under vacuum and purged with argon and then flushed with hydrogen gas to expel the argon. The mixture was stirred at rt under H₂-atmosphere (Balloon-pressure) for 20 h then flushed with argon gas to expel the hydrogen. The mixture was filtered over a short pad of silica, washing with EtOAc (2×100 ml) and the filtrate was concentrated under reduced pressure to give 8,8′-oxybis(octan-1-ol) (11.05 g, 40.2 mmol, 96% yield) as a beige solid.

1H NMR (400 MHz, CDCl3, 298 K) δ (ppm)=3.62 (t, J=6.7 Hz, 4H), 3.39 (t, J=6.7 Hz, 4H), 1.95 (br s, 2H), 1.64-1.48 (m, 8H), 1.41-1.26 (m, 16H).

13C NMR (101 MHz, CDCl3, 298 K) δ (ppm)=70.9, 62.9, 32.7, 29.7, 29.4, 29.4, 26.1, 25.7. GC/MS (El, 70 eV): 256 (1, [M-H₂O]⁺*), 145 (17), 129 (6), 127 (14), 109 (46), 69 (100).

Example 3d: 8,8′-oxydioctanoic acid: A solution of 8,8′-oxybis(octan-1-ol) (10.0 g, 36.4 mmol) in Acetone (200 mL) was treated dropwise at r.t. (water bath cooling) with Jones reagent (67 g, 52 mL, 36.4 mmol) over 60 min until the reaction mixture showed a persistent orange color. After stirring for 2 h at r.t., the reaction mixture was quenched with isopropanol (50 ml).

The reaction mixture was poured into ice cold NaOH 2M (150 ml) and stirred vigorously for 10 min. The mixture was extracted with EtOAc (1×150 ml) and the org. layer was discarded.

The aq. layer was then acidified with HCl 5 M to pH 1, filtered, extracted with CH₂Cl₂ (2×200 ml) and the org. layers were washed with brine (1×150 ml). The combined org. layers were dried over Na₂SO₄, filtered and concentrated under reduced pressure to give 8,8′-oxydioctanoic acid (9.40 g, 80% purity, 25 mmol, 69% yield) as a white solid which was used without further purification.

1H NMR (400 MHz, CDCl3, 298 K) δ (ppm)=10.29 (br s, 2H), 3.40 (t, J=6.7 Hz, 4H), 2.34 (t, J=7.5 Hz, 4H), 1.72-1.49 (m, 8H), 1.44-1.23 (m, 12H).

13C NMR (101 MHz, CDCl3, 298 K) δ (ppm)=180.1, 70.8, 34.1, 29.6, 29.0, 29.0, 26.0, 24.6.

Example 3e: dimethyl 8,8′-oxydioctanoate: A mixture of 8,8′-oxydioctanoic acid (9.40 g, 31.1 mmol), Methanol (200 mL) and 1 drop of sulfuric acid (311 mg, 0.169 mL, 3.11 mmol) was heated to 65° C. and stirred for 3.5 h. The reaction mixture was poured into ice water (200 ml) and stirred vigorously for 10 min. The mixture was extracted with MTBE (2×150 ml), the org. layers were washed with water (1×150 ml), aqueous saturated NaHCO₃ solution (1×100 ml) and brine (1×100 ml). The combined org. layers were dried over Na₂SO₄, filtered and concentrated under reduced pressure to give dimethyl 8,8′-oxydioctanoate (9.40 g, 95% purity, 29.2 mmol, 94% yield) as a pale yellow liquid.

1H NMR (400 MHz, CDCl3, 298 K) δ (ppm)=3.66 (s, 6H), 3.38 (t, J=6.7 Hz, 4H), 2.30 (t, J=7.5 Hz, 4H), 1.70-1.49 (m, 8H), 1.40-1.26 (m, 12H).

13C NMR (101 MHz, CDCl3, 298 K) δ (ppm)=174.2, 70.9, 51.4, 34.0, 29.7, 29.1, 29.1, 26.0, 24.9.

GC/MS (El, 70 eV): 299 (1, [M-MeO]⁺*), 281 (1), 267 (1), 257 (3), 173 (48), 157 (26), 141 (100), 125 (80), 59 (19), 55 (69).

Example 3f: methyl 9-oxooxacyclohexadecane-8-carboxylate: A solution of dimethyl 8,8′-oxydioctanoate (5.00 g, 15.1 mmol) in THF (270 ml) was added over 6 h under N₂ atmosphere to a gently refluxing mixture of NaHMDS (27.7 g, 75.6 mL, 2.0 molar in THF, 151 mmol) and THF (450 ml). After the addition the reaction mixture was refluxed for an additional 1 h then cooled to r.t. and treated slowly with acetic acid (50 ml). The reaction mixture was washed with water (2×250 ml) and the water layers were extracted with MTBE (250 ml). The org. layers were finally washed with brine (1×200 ml), dried over Na₂SO₄, filtered and concentrated under reduced pressure. The resulting material was purified by column chromatography on silica gel, eluting with a gradient of MTBE in heptane to give methyl 9-oxooxacyclohexadecane-8-carboxylate (1.60 g, 96% purity, 4.90 mmol, 32% yield) as a pale yellow liquid.

1H NMR (400 MHz, CDCl3, 298 K) δ (ppm)=3.77-3.65 (m, 3H), 3.57 (dd, J=6.5, 8.4 Hz, 1H), 3.51-3.35 (m, 4H), 2.64-2.47 (m, 2H), 2.05-1.79 (m, 2H), 1.78-1.19 (m, 18H).

13C NMR (101 MHz, CDCl3, 298 K) δ (ppm)=206.6, 170.2, 69.4, 69.4, 57.6, 52.2, 40.7, 29.0, 28.9, 27.8, 27.6, 27.5, 27.3, 26.6, 25.5, 25.4, 22.8.

GC/MS (El, 70 eV): 298 (2, [M]+*), 283 (1), 280 (1), 267 (9), 248 (2), 238 (1), 125 (58), 98 (46), 87 (34), 55 (100), 41 (37).

Example 3g: oxacyclohexadecan-9-one: A solution of methyl 9-oxooxacyclohexadecane-8-carboxylate (1.6 g, 5.4 mmol) in MeOH (50 ml) was treated with aqueous NaOH (0.64 g, 8.0 mL, 2 molar, 16 mmol) and the resulting mixture heated to reflux (80° C.) for 1 h. The resulting mixture was then cooled to r.t., acidified to pH 1 by dropwise addition of 10% H₂SO₄ and then heated to re-flux for 30 min. The mixture was then cooled to r.t., extracted with MTBE (2×100 mL), washed with water (100 mL), brine (100 mL), dried over Na₂SO₄, filtered and concentrated. The resulting material was purified by flash column chromatography on silica gel, eluting with a gradient of MTBE in heptane to give oxacyclohexadecan-9-one (1.0 g, 4.2 mmol, 77% yield) as a pale yellow liquid.

1H NMR (400 MHz, CDCl3, 298 K) δ (ppm)=3.48-3.38 (m, 4H), 2.41 (t, J=7.2 Hz, 4H), 1.74-1.62 (m, 4H), 1.60-1.50 (m, 4H), 1.49-1.39 (m, 4H), 1.38-1.26 (m, 8H).

13C NMR (101 MHz, CDCl3, 298 K) δ (ppm)=212.8, 69.4, 41.5, 29.0, 27.9, 27.3, 25.4, 23.5. GC/MS (El, 70 eV): 240 (5, [M]⁺*), 225 (1), 197 (1), 153 (11), 125 (45), 97 (45), 82 (58), 55 (100), 41 (54), 29 (15).

Odour description (10% solution in DPG on paper blotter, 4 h): musky, powdery, nitro musk, warm.

### Example 4a to 4e

Following the general procedure described in Example 3, the compounds 4a-4e were prepared.

4a: oxacyclohexadecan-8-one: using 9-(phenylmethoxy)-1-nonanol in the first step (Tosylation), then reacting it with 7-(phenylmethoxy)-1-heptanol, followed by the steps described. This gave oxacyclohexadecan-8-one as a pale yellow oil.

¹H NMR (400 MHz, CDCl₃) δ (ppm)=3.44-3.23 (m, 4H), 2.52-2.18 (m, 4H), 1.72-1.52 (m, 4H), 1.52-1.39 (m, 4H), 1.38-1.15 (m, 12H).

¹³C NMR (101 MHz, CDCl₃) δ (ppm)=211.59 (q), 68.99 (t), 68.64 (t), 41.02 (t), 40.64 (t), 28.30 (t), 27.96 (t), 27.08 (t), 26.51 (t), 26.21 (t), 26.09 (t), 25.03 (t), 24.04 (t), 22.64 (t), 22.36 (t). GC/MS (El): m/z (%): 240 (5) [M+], 139 (17), 126 (18), 111 (28), 97 (26), 83 (33), 81 (25), 69 (45), 55 (100).

Odour description (10% solution in DPG on paper blotter, 4 h): musky, powdery, nitro musk, warm.

4b: oxacycloheptadecan-7-one: using 6-(benzyloxy)-1-hexanol in the first step (Tosylation), then reacting it with 11-(phenylmethoxy)-1-undecanol, followed by the steps described. This gave oxacycloheptadecan-7-one as a colorless liquid.

¹H NMR (500 MHz, CDCl3, 298 K) δ (ppm)=3.45-3.37 (m, 4H), 2.43 (t, J=6.7 Hz, 2H), 2.40 (t, J=6.9 Hz, 2H), 1.68-1.59 (m, 4H), 1.58-1.49 (m, 4H), 1.45-1.35 (m, 4H), 1.35-1.23 (m, 10H).

¹³C NMR (126 MHz, CDCl3, 298 K) δ (ppm)=212.4, 70.3, 70.2, 42.5, 42.5, 29.8, 29.2, 27.9, 27.9, 27.5, 27.2, 27.1, 26.6, 25.6, 24.1, 23.3.

GC/MS (El, 70 eV): 254 (5, [M]+*), 236 (1), 211 (1), 125 (23), 112 (26), 97 (35), 83 (21), 69 (48), 55 (100), 41 (70).

Odour description (10% solution in DPG on paper blotter, 4 h): musky, powdery, nitro musk, slightly ambery.

4c: oxacycloheptadecan-6-one: using 5-(Benzyloxy)-1-pentanol in the first step (Tosylation), then reacting it with 12-(Phenylmethoxy)-1-dodecanol, followed by the steps described. This gave oxacycloheptadecan-6-one as a colorless liquid.

¹H NMR (500 MHz, CDCl3, 298 K) δ (ppm)=3.47-3.39 (m, 4H), 2.49-2.39 (m, 4H), 1.77-1.69 (m, 2H), 1.68-1.58 (m, 4H), 1.58-1.51 (m, 2H), 1.47-1.38 (m, 2H), 1.37-1.26 (m, 12H).

¹³C NMR (126 MHz, CDCl3, 298 K) δ (ppm)=212.7, 70.3, 43.1, 41.9, 29.4, 29.4, 27.6, 27.3, 27.2, 26.9, 25.5, 23.6, 22.1.

GC/MS (El, 70 eV): 254 (5, [M]⁺*), 236 (1), 225 (1), 111 (32), 98 (82), 83 (60), 69 (22), 55 (100), 41 (65).

Odour description (10% solution in DPG on paper blotter, 4 h): musky, powdery, nitro musk, slightly ambery.

4d: oxacycloheptadecan-9-one: using 8-(Benzyloxy)-1-octanol in the first step (Tosylation), then reacting it with 9-(Phenylmethoxy)-1-nonanol, followed by the steps described. This gave oxacycloheptadecan-9-one as a colorless liquid.

¹H NMR (500 MHz, CDCl3, 298 K) δ (ppm)=3.46-3.37 (m, 4H), 2.47-2.35 (m, 4H), 1.70-1.59 (m, 4H), 1.58-1.50 (m, 4H), 1.47-1.38 (m, 4H), 1.38-1.22 (m, 10H).

¹³C NMR (126 MHz, CDCl3, 298 K) δ (ppm)=212.9, 70.0, 69.9, 42.9, 41.9, 29.5, 29.4, 28.7, 28.3, 28.2, 28.2, 25.8, 24.1, 23.8.

GC/MS (El, 70 eV): 254 (3, [M]⁺*), 239 (1), 211 (1), 197 (1), 171 (2), 153 (4), 139 (9), 125 (15), 111 (14), 97 (26), 82 (24), 69 (24), 55 (100), 41 (55).

Odour description (10% solution in DPG on paper blotter, 4 h): musky, powdery, nitro musk, slightly ambery, warm.

4e: oxacycloheptadecan-8-one: using 7-(Benzyloxy)-1-heptanol in the first step (Tosylation), then reacting it with 10-(Phenylmethoxy)-1-decanol, followed by the steps described. This gave oxacycloheptadecan-8-one as a colorless liquid.

¹H NMR (500 MHz, CDCl3, 298 K) δ (ppm)=3.47-3.38 (m, 4H), 2.44 (t, J=6.9 Hz, 2H), 2.42-2.38 (m, 2H), 1.71-1.61 (m, 4H), 1.59-1.51 (m, 4H), 1.46-1.24 (m, 14H).

¹³C NMR (126 MHz, CDCl3, 298 K) δ (ppm)=212.4, 70.2, 70.1, 42.1, 41.9, 29.4, 29.3, 28.4, 28.1, 28.0, 27.8, 27.4, 26.0, 25.9, 23.7, 23.5.

GC/MS (El, 70 eV): 254 (4, [M]+*), 236 (1), 211 (1), 197 (2), 186 (3), 169 (2), 153 (3), 139 (9), 126 (16), 111 (22), 98 (15), 83 (24), 69 (36), 55 (100), 41 (58).

Odour description (10% solution in DPG on paper blotter, 4 h): musky, powdery, nitro musk, slightly ambery.

### Example 5: Fragrance Composition

| | parts |
|---|---|
| | by |
| Ingredient | weight |
| Benzyl acetate | 0.6 |
| 3,7-Dimethyl-1,6-octadien-3-yl acetate | 0.5 |
| 2-Methoxy-4-(1-propenyl)phenyl acetate | 0.1 |
| 2-Phenylethanol | 2.0 |
| 2-Hexyl-3-phenyl-2-propenal | 0.3 |
| Decanal @ 10% in TEC | 0.4 |
| Dodecanal @ 10% in TEC | 0.5 |
| Dipropylene glycol (DPG) | 62.9 |
| Cinnamaldehyde @ 10% in TEC | 0.4 |
| Ambrox ™ | 0.2 |
| Bergamot oil | 6.0 |
| Cedarwood oil | 0.1 |
| Ethylene Brassylate | 14.0 |
| Cashmeran ™ (6,7-dihydro-1,1,2,3,3-pentamethyl-4(5h)- | 0.1 |
| indanone | |
| Citronellol (3,7-dimethyl-6-octen-1-ol) | 0.3 |
| Coumarine pure | 0.7 |
| Gardenol ™ (1-phenylethyl acetate) | 0.1 |
| Geraniol | 0.4 |
| Hedione ™ (methyl 3-oxo-2-pentylcyclopentaneacetate | 0.7 |
| 7-Hydroxy-3,7-dimethyloctan-1-al | 2.3 |
| Indole @ 1% in TEC | 1.0 |
| Iris absolute | 0.2 |
| Isoraldeine ® | 2.5 |
| cis-Jasmone (3-methyl-2-(pent-2-en-1-yl)cyclopent-2-enone) | 0.1 |
| @ 10% in DPG | |
| Linalol | 0.6 |
| Methyl 2-(methylamino)benzoate @ 10% in DPG | 0.7 |
| Methyl cedryl ketone | 0.2 |
| Nerolex ™ (3,7-dimethyl-2,6-octadien-1-ol) | 0.2 |
| sweet orange oil | 1.0 |
| Patchouli oil | 0.2 |
| Peche Pure ™ (4-undecanolide) @ 10% in DPG | 0.2 |
| Phenylethyl phenylacetate | 0.2 |
| Vetiver oil | 0.3 |
| | 100.0 |

By replacing 8 parts DPG with 8 parts oxacyclohexadecan-9-one (a compound of formula (I)) the fragrance composition is perceived more rounded off, better blended, warmer with a musky note, adding depth to the fragrance.

1. A method of providing fragrance, the method comprising adding a compound of formula (I) 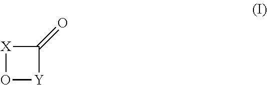
wherein
X is a divalent residue —(CH2)ₙ— optionally substituted with one additional methyl group,
Y is divalent residue —(CH2)ₘ—,
n is an integer selected from 3 to 12, and
m is an integer selected from 3 to 12,
with the proviso that the n+m is at least 13 and not greater than 15; and
with the proviso that the compound of formula (I) is not oxacyclohexadecan-6-one to at least one other odorant or to a consumer product base.

2. A fragrance composition comprising a compound of formula (I) 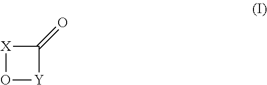
wherein
X is a divalent residue —(CH2)ₙ— optionally substituted with one additional methyl group,
Y is divalent residue —(CH2)ₘ—,
n is an integer selected from 3 to 12, and
m is an integer selected from 3 to 12,
with the proviso that the n+m is at least 13 and not greater than 15; and
at least one other odorant.

3. A fragrance composition comprising a compound according to claim 1, wherein the compound is selected from the group consisting of 4-methyloxacyclopentadecan-6-one; 3-methyloxacyclopentadecan-5-one; oxacyclopentadecan-5-one; oxacyclopentadecan-6-one; oxacyclopentadecan-7-one; oxacyclopentadecan-8-one; 3-methyloxacyclohexadecan-5-one; 4-methyloxacyclohexadecan-6-one; oxacyclohexadecan-5-one; oxacyclohexadecan-7-one; oxacyclohexadecan-8-one; oxacycloheptadecan-5-one; oxacycloheptadecan-6-one; oxacycloheptadecan-7-one; oxacycloheptadecan-8-one; oxacycloheptadecan-9-one and combinations thereof.

4. A fragranced article comprising
a) as odorant a compound of formula (I) 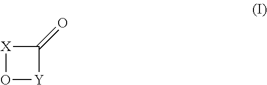
wherein
X is a divalent residue —(CH2)— optionally substituted with one additional methyl group,
Y is divalent residue —(CH2)ₘ—,
n is an integer selected from 3 to 12, and
m is an integer selected from 3 to 12,
with the proviso that the n+m is at least 13 and not greater than 15; and
b) a consumer product base.

5. The fragranced article according to claim 4 wherein the consumer product base is selected from fine perfumery, personal care products and fabric care products.

6. A method of improving, enhancing or modifying a consumer product base by means of addition thereto of an olfactory acceptable amount of a compound of formula (I) as defined in claim 1.

7. A compound of formula (I) 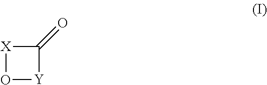
wherein
X is a divalent residue —(CH2)ₙ— optionally substituted with one additional methyl group,
Y is divalent residue —(CH2)ₘ—,
n is an integer selected from 3 to 7, and
m is an integer selected from 8 to 11,
with the proviso that the n+m is at least 13 and not greater than 15; and
with the proviso that oxacyclohexadecan-6-one and oxacyclohexadecan-5-one are excluded.

8. A compound according to claim 6 selected from the group consisting of 4-methyloxacyclopentadecan-6-one; 3-methyloxacyclopentadecan-5-one; oxacyclopentadecan-5-one; oxacyclopentadecan-6-one; oxacyclopentadecan-7-one; oxacyclopentadecan-8-one; 3-methyloxacyclohexadecan-5-one; 4-methyloxacyclohexadecan-6-one; oxacyclohexadecan-7-one; oxacyclohexadecan-8-one; oxacycloheptadecan-5-one; oxacycloheptadecan-6-one; oxacycloheptadecan-7-one; oxacycloheptadecan-8-one; oxacycloheptadecan-9-one, and combinations thereof.

9. The method according to claim 1, wherein
X is a divalent residue —(CH2)ₙ— substituted with one additional methyl group,
Y is divalent residue —(CH2)ₘ—,
n is an integer selected from 3 and 4, and
m is an integer selected from 9 to 11,
with the proviso that the n+m is at least 13 and not greater than 15.

10. The method according to claim 1, wherein
X is a divalent residue —(CH2)ₙ—,
Y is divalent residue —(CH2)ₘ—,
n is an integer selected from 3 to 7, and
m is an integer selected from 7 to 12,
with the proviso that the n+m is at least 13 and not greater than 15; and
with the proviso that the compound of formula (I) is not oxacyclohexadecan-6-one.

11. The method according to claim 1, wherein
X is a divalent residue —(CH2)ₙ—,
Y is divalent residue —(CH2)ₘ—,
n is an integer selected from 6, 7 and 8, and
m is an integer selected from 6, 7 and 8,
with the proviso that n+m is at least 13 and not greater than 15.

12. The method according to claim 1, wherein
X is a divalent residue —(CH2)ₙ—,
Y is divalent residue —(CH2)ₘ—,
n is an integer selected from 6 and 7, and
m is an integer selected from 6 to 9,
with the proviso that n+m is at least 13 and not greater than 15.

13. The method according to claim 1, wherein the compound of formula (I) is selected from the group consisting of 4-methyloxacyclopentadecan-6-one; 3-methyloxacyclopentadecan-5-one; oxacyclopentadecan-5-one; oxacyclopentadecan-6-one; oxacyclopentadecan-7-one; oxacyclopentadecan-8-one; 3-methyloxacyclohexadecan-5-one; 4-methyloxacyclohexadecan-6-one; oxacyclohexadecan-5-one; oxacyclohexadecan-7-one; oxacyclohexadecan-8-one; oxacycloheptadecan-5-one; oxacycloheptadecan-6-one; oxacycloheptadecan-7-one; oxacycloheptadecan-8-one; oxacycloheptadecan-9-one, and combinations thereof.
